(19) United States
(12) Patent Application Publication
(10) Pub. No.: US 2005/0074494 A1
(43) Pub. Date: Apr. 7, 2005

(54) Itraconazole immediate release formulation

(76) Inventors: Xiu-Xiu Cheng, Weston, FL (US); Chih Ming Chen, Taipei (TW); Steve Jan, Coral Springs, FL (US)

Correspondence Address: HEDMAN & COSTIGAN P.C., 1185 AVENUE OF THE AMERICAS, NEW YORK, NY, 10036, US

(21) Appl. No.: 10/679,760

(22) Filed: Oct. 6, 2003

(51) Int. Cl. A61K 9/24; A61K 9/16; A61K 9/50; A61K 31/496

(52) U.S. Cl. 424/471; 514/254.07

(57) ABSTRACT

The present invention relates to active pellets without a specific inert starting seed size and without a seal coat, which may be compressed into a tablet or loaded into a capsule to form an orally administrable dosage formulation for an antifungal agent.

### FIELD OF THE INVENTION

The present invention relates to the field of oral dosage forms. More particularly, it relates to an immediate release oral dosage form of an antifungal for the treatment in mammals of fungi and related ailments.

### BACKGROUND OF THE INVENTION

There exists a need for an orally administrable antifungal medication with a high concentration of active ingredient that can be manufactured in an economically feasible manner. U.S. Pat. No. 5,633,015 (incorporated herein by reference) describes oral pharmaceutical formulations containing itraconazole beads that are seal coated. Furthermore, a specific core size must be used to make the beads. A seal coat is generally employed for protection of the active ingredient or drug during processing and to reduce unwanted reactions. However, a seal coat also increases manufacturing time and costs.

Antifungal agents have been shown to be advantageous in the treatment of nail and skin fungus infections. Antifungal compositions are well known in the art. Specifically, U.S. Pat. No. 4,267,179, which describes the synthetic triazole antifungal compound itraconazole, 4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one, molecular formula C₃₅H₃₈Cl₂N₈O₄; and U.S. Pat. No. 4,916,134, which describes itraconazole's difluro analog, saperconazole, 4-[4-[4-[4-[[2-(2,4-difluorophenyl)-2(1H-1,2,4-triazol-1-ylmethyl)-1,3-dioxolan-4-yl]methoxy]phenyl]-1-piperazinyl]-phenyl]-2,4-dihydro-2-(1-methoxypropyl)-3H-1,2,4-triazol-3-one, molecular formula C₃₅H₃₈F₂N₈O₄ (see The Merck Index p. 5263 and 8435, 13^{th} Ed. (2001) and Physicians' Desk Reference 1313-1315, 38^{th} Ed. (1998)). Both patents are incorporated herein by reference.

Itraconazole has a molecular formula of C₃₅H₃₈Cl₂N₈O₄ and a molecular weight of 705.64. It is insoluble in water, very slightly soluble in alcohols and freely soluble in dichloromethane. Additionally, it has a pKa of 3.70 (based on extrapolation of values obtained form methanolic solutions).

The drug itraconazole is commercially available in capsules, injections, and oral solutions (SPORANOX®). See Physician Desk Reference 55^{th} Ed., pp. 1584-94 (which is incorporated herein by reference).

### SUMMARY OF THE INVENTION

The present invention overcomes the drawbacks of the prior art through the novel development of an oral antifungal dosage form without the need of a seal coat, and without the limitation of a specific starting seed size. The lack of a seal coat substantially reduces manufacturing time and costs.

The foregoing advantages are obtained by a preparing an active pellet comprising:
a) an inert starting seed;
b) an anti-fungal agent;
c) a binder; and optionally
d) an alkaline agent.

The active pellets without a seal coat can be mixed with conventional tabletting excipients and compressed into a tablet or loaded into a capsule for oral administration.

The present invention also relates to a method of producing the pellets or beads.

In a preferred embodiment of the present invention an organic solvent system is used to apply the drug layer to the inert starting seed.

### DETAILED DESCRIPTION OF THE INVENTION

In the present invention, the inert starting seed is a sugar seed with a mesh size of 15-40, preferably 18-20. The inert starting seed must be of sufficient density and strength to enable it to undergo coating in a fluidized bed process. Suitable starting seeds are sugar seeds or non-pariels that are well known in the art. Additional suitable starting seeds may be chosen from plastic resins, silica, glass, microcrystalline cellulose, hydroxyapatite, sodium chloride, potassium chloride, calcium carbonate, magnesium carbonate, activated carbon, citric acid, fumaric acid, tartaric acid, ascorbic acid, oligosaccharides, glucose, rhamnose, galactose, lactose, sucrose, mannitol, sorbitol, dextrin, maltodextrin, cellulose, sodium carboxymethyl cellulose and starch. The preferred starting seed is a sugar sphere with a mesh size of 18-20.

The pharmaceutically active ingredient or drug that is applied to the inert starting seed is itraconazole or its difluoro analog, saperconazole.

In order for the drug to be applied to the inert starting seed, a binding agent may be necessary. The binding agent employed in preparation of the active pellet in accordance with the present invention can be any type of binding agent commonly known in the art. Examples of some of the preferred binding agents are polyvinyl pyrrolidone, hydroxyethyl cellulose, hydroxypropyl cellulose, hydroxypropyl methylcellulose, polyacrylate, ethylcellulose, or mixtures of the foregoing. In the preferred embodiment of the present invention, the binding agent is hydroxypropyl methylcellulose (available as METHOCEL® E5).

The drug is applied to the inert starting seed by any conventional techniques known in the industry, such as, pan coating, roto-granulation or fluidized bed coating. During such coating operations the drug is dispersed or dissolved in an organic solvent, which may also contain other conventional excipients, such as the above mentioned binding agent.

Representative examples of the alkaline agent that may be used in preparation of the present invention are amino acids, such as lysine, arginine, ornitine, histidine, organic buffering compounds such as trometamine (i.e. Tris-buffer), N-amino sugars such as N-methyl-D-glucamine (i.e. Meglumine), N-ethyl-D-glucamine (i.e. Eglumine), glucosamine, disodium-N-stearoyl-glutamate, heterocyclic amine derivatives such as piperazine or its hexahydrate, N-methylpiperazine, morpholine, 1-(2-hydroxyethyl)pyrrolidine, alkali salts of citric acid, tartaric acid, caproic acid or fatty acids, alkali metal phosphates, silicates or carbonates, sodium, potassium, magnesium, calcium or aluminium hydroxides and organic amines such as ethylamine, dicyclohexylamine or triethanolamine, or alkaline ammonium salts.

The weight percentages of ingredients in the present invention based on the final pellet composition can be seen in the following table.

TABLE I

| Ingredients | Preferred | Most preferred |
|---|---|---|
| Inert seed | At least 35% | 35-55% |
| drug | 10-50% | 15-40% |
| binder | 10-50% | 25-40% |
| alkaline agent | 0-5% | 0-3% |

The tablet or capsule containing the active pellets prepared in accordance with the present invention should obtain its peak plasma level within about 3 to 9 hours, preferably about 3.5 hours to about 6.5 hours and have a Cₘₐₓ of less than 100 ng/ml, preferably less than 90 ng/ml, and most preferably between 40 ng/ml and 80 ng/ml.

### DESCRIPTION OF THE PREFERRED EMBODIMENT

The present invention will be further illustrated by the following examples.

### EXAMPLE I

An immediate release itraconazole capsule in accordance with the present invention is prepared as follows.

Stage I Drug Solution

70.25 kg of methylene chloride is placed in a 30-gallon stainless-steel tank with 46.83 kg of Ethanol SDA 3A 190 Proof. Next, 6.109 kg of hydroxypropyl methylcelluose (Methocel® E5) is added into the previously formed organic solvent and mixed for at least 30 minutes. Subsequently, 4.072 kg of itraconazole is added to the above mixture and mixed for at least 20 minutes.

Stage II Spray Coating

7.819 kg of sugar spheres NF 18/20 are placed into a fluidized bed coater. The sugar spheres should be preheated for 3 minutes at an inlet air temperature of 55° C.±5° C. The drug suspension prepared above is sprayed onto the sugar seeds under the following conditions:

TABLE II

| | |
|---|---|
| Fluidized Bed Coater | Bottom Spray with Wurster insert |
| Nozzle tip diameter | 1.8 mm |
| Shaking interval | 1 min |
| Shaking Duration | 3 sec |
| Atomization Pressure | 2-3 bar |
| Inlet Air Temperature | 55-60° C. |
| Pump Rate | 15-150 mL/min |
| Tubing Size | 24 mm |

The drug solution should be sprayed slowly at the beginning to avoid agglomeration of the sugar spheres. As the spraying continues the rate of drug application can be increased.

Once the drug suspension has been consumed the pellets are dried for 20 minutes or until the loss on drying (LOD) is less than 1%. The pellets are then placed on trays in one-half inch layers and dried in an oven at 75° C. for at least 40 hours.

Stage III Encapsulation

The itraconazole pellets are dusted with 0.360 kg of talc and encapsulated utilizing equipment and guidelines commonly known in the art. Natural/Aqua blue opaque size “0” capsules are filled with itraconazole pellets, which contain 100 mg of itraconazole.

### EXAMPLE II

An immediate release itraconazole capsule in accordance with the present invention is prepared as follows.

Stage I Drug Solution

70.25 kg of methylene chloride is placed in a steel 30-gallon drum with 46.83 kg of Ethanol SDA 3A 190 Proof. Next, 6.109 kg of hydroxypropyl methylcelluose (Methocel® E5) is added into the previously formed organic solvent and mixed for at least 30 minutes. Subsequently, 4.072 kg of itraconazole is added to the above mixture and mixed for at least 20 minutes. 0.081 kg of L-arginine base is added into 0.459 kg of purified water and mixed for 20 minutes and then added to the above mixture.

Stage II Spray Coating

7.738 kg of sugar spheres NF 18/20 are placed into a fluidized bed coater. The sugar spheres should be preheated for 3 minutes at an inlet air temperature of 55° C.±5° C. The drug suspension prepared above is sprayed onto the sugar seeds using the conditions and parameters described in Example I.

Once the drug suspension has been consumed the pellets are dried for 20 minutes or until the loss on drying (LOD) is less than 1%. The pellets are then placed on trays in one-half inch layers and dried in an oven at 75° C. for at least 40 hours.

Stage III Encapsulation

The itraconazole pellets are dusted with 0.360 kg of talc and encapsulated utilizing the equipment and guidelines commonly known in the art. Natural/Aqua blue opaque size “0” capsules are filled with itraconazole pellets, which contain 100 mg of itraconazole.

### EXAMPLE III

An immediate release itraconazole capsule in accordance with the present invention is prepared as follows.

Stage I Drug Solution

70.25 kg of methylene chloride is placed in a steel 30-gallon drum with 46.83 kg of Ethanol SDA 3A 190 Proof. Next, 6.109 kg of hydroxypropyl methylcelluose (Methocel® E5) is added into the previously formed organic solvent and mixed for at least 30 minutes. Subsequently, 4.072 kg of itraconazole is added to the above mixture and mixed for at least 20 minutes.

Stage II Spray Coating

7.819 kg of sugar spheres NF 25/30 are placed into a fluidized bed coater. The sugar spheres should be preheated for 3 minutes at an inlet air temperature of 55° C.±5° C. The drug suspension prepared above is sprayed onto the sugar seeds using the conditions and parameters described in Example I.

Once the drug suspension has been consumed the pellets are dried for 20 minutes or until the loss on drying (LOD) is less than 1%. The pellets are then placed on trays in one-half inch layers and dried in an oven at 75° C. for at least 40 hours.

Stage III Encapsulation

The itraconazole pellets are encapsulated utilizing the equipment and guidelines commonly known in the art. Natural/Aqua blue opaque size “0” capsules are filled with itraconazole pellets, which contain 100 mg of itraconazole.

A biostudy between the above product and the SPORANOX® reference product using 29 individuals under fasting conditions produced the following results:

TABLE III

| | Test | | Ref | | G-mean | 90% | 90% | |
|---|---|---|---|---|---|---|---|---|
| | Mean | % CV | Mean | % CV | Ratio | C.I. | C.I. | Mid |
| Cmax | 51.13 | 69.21 | 37.20 | 65.96 | 1.328 | 109.00% | 162.27% | 135.64% |
| AUC 0˜t | 586.86 | 70.90 | 433.70 | 66.69 | 1.319 | 110.73% | 156.91% | 133.82% |
| AUC 0˜inf | 644.09 | 71.93 | 477.48 | 65.58 | 1.313 | 110.99% | 155.06% | 133.02% |
| Tmax | 3.86 | 26.55 | 3.72 | 25.77 | 1.034 | | | |

A biostudy using 19 individuals between the above product under fed and fasting conditions and the SPORANOX® reference product under fed conditions produced the following results:

TABLE IV

| Test Fed/ | Test | | Ref | | G-mean | A-mean |
|---|---|---|---|---|---|---|
| Reference Fed | Mean | % CV | Mean | % CV | Ratio | Ratio |
| Cmax | 70.13 | 49.01 | 57.35 | 42.72 | 1.174 | 1.409 |
| AUC 0˜t | 709.17 | 41.75 | 561.18 | 45.68 | 1.274 | 1.452 |
| AUC 0˜inf | 745.97 | 41.36 | 594.83 | 45.91 | 1.265 | 1.436 |
| Tmax | 5.26 | 8.60 | 5.21 | 10.27 | 1.012 | 1.017 |

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof which do not depart from the spirit and scope of the invention.

1. An active pellet consisting essentially of:
A) an inert starting seed;
B) an antifungal agent;
C) a binder; and
d) optionally an alkaline agent.

2. The active pellet according to claim 1, wherein the inert starting seed has a mesh size of 15-40.

3. The active pellets according to claim 1, wherein the inert starting seed has a mesh size of 18-20.

4. The active pellets according to claim 1, wherein the inert starting seeds are selected from the group consisting of plastic resins, silica, glass, microcrystalline cellulose, hydroxyapatite, sodium chloride, potassium chloride, calcium carbonate, magnesium carbonate, activated carbon, citric acid, fumaric acid, tartaric acid, ascorbic acid, oligosaccharides, glucose, rhamnose, galactose, lactose, sucrose, mannitol, sorbitol, dextrin, maltodextrin, cellulose, sodium carboxymethyl cellulose and starch.

5. The active pellets according to claim 2, wherein the inert starting seed is a sugar sphere.

6. The active pellets according to claim 2, wherein the inert starting seed is microcrystalline cellulose.

7. The active pellets according to claim 2, wherein the antifungal is itraconazole.

8. The active pellets according to claim 2, wherein the binder is selected from the group consisting of polyvinyl pyrrolidone, hydroxyethyl cellulose, hydroxypropyl cellulose, Hydroxypropyl methylcellulose, polyacrylate, ethylcellulose, or mixtures thereof.

9. The active pellets, according to claim 7, wherein the binder is hydroxypropyl methylcellulose.

10. A pharmaceutical composition comprising a capsule and a plurality of active pellets as defined in claim 1.

11. An active pellet consisting essentially of:
A) at least 35% by weight of a inert starting seed;
B) 10-50% by weight of an antifungal agent;
C) 10-50% by weight of a binder; and
D) 0-5% by weight of an alkaline agent.

12. An active pellet, according to claim 11, consisting essentially of:
A) 35-55% by weight of an inert starting seed;
B) 15-40% by weight of an antifungal agent;
C) 25-40% by weight of a binder; and
D) 0-3% by weight of an alkaline agent.

13. The active pellet according to claim 11, wherein the inert starting seed has a mesh size of 15-40.

14. The active pellets according to claim 11, wherein the inert starting seed has a mesh size of 18-20.

15. The active pellets according to claim 11, wherein the inert starting seeds are selected from the group consisting of plastic resins, silica, glass, microcrystalline cellulose, hydroxyapatite, sodium chloride, potassium chloride, calcium carbonate, magnesium carbonate, activated carbon, citric acid, fumaric acid, tartaric acid, ascorbic acid, oligosaccharides, glucose, rhamnose, galactose, lactose, sucrose, mannitol, sorbitol, dextrin, maltodextrin, cellulose, sodium carboxymethyl cellulose and starch.

16. The active pellets according to claim 13, wherein the inert starting seed is a sugar sphere.

17. The active pellets according to claim 13, wherein the inert starting seed is microcrystalline cellulose.

18. The active pellets according to claim 13, wherein the antifungal is itraconazole.

19. The active pellets according to claim 13, wherein the binder is selected from the group consisting of polyvinyl pyrrolidone, hydroxyethyl cellulose, hydroxypropyl cellulose, Hydroxypropyl methylcellulose, polyacrylate, ethylcellulose, or mixtures thereof.

20. The active pellets, according to claim 19, wherein the binder is hydroxypropyl methylcellulose.

21. A pharmaceutical composition comprising a capsule and a plurality of active pellets as defined in claim 11.

22. The pharmaceutical capsule as defined in claim 10 that exhibits a peak plasma level between 3 and 9 hours after administration.

23. The pharmaceutical capsule as defined in claim 10 that exhibits a Cₘₐₓ of less than 100 ng/ml.

24. The pharmaceutical capsule as defined in claim 10 that exhibits a Cₘₐₓ of less than 90 ng/ml.

25. The pharmaceutical capsule as defined in claim 10 that exhibits a Cₘₐₓ of between 40 ng/ml and 80 ng/ml.

27. The pharmaceutical capsule as defined in claim 11 that exhibits a peak plasma level between 3 and 9 hours after administration.

28. The pharmaceutical capsule as defined in claim 11 that exhibits a Cₘₐₓ of less than 100 ng/ml.

29. The pharmaceutical capsule as defined in claim 11 that exhibits a Cₘₐₓ of less than 90 ng/ml.

30. The pharmaceutical capsule as defined in claim 11 that exhibits a Cₘₐₓ of between 40 ng/ml and 80 ng/ml.

31. An antifungal pharmaceutical dosage form for oral administration consisting essentially of:
a) a geletin capsule; and
b) a plurality of active pellets, wherein each pellet consists essentially of:
i) at least 40% by weight of an 18-20 mesh sugar sphere.
ii) 10-50% by weight of itraconazole;
iii) 10-50% by weight of a binder; and
iv) 0-5% by weight of an alkaline agent.

32. A pharmaceutical tablet comprising a plurality of active pellets as defined in claim 1 and convention tabletting excipients.

33. A pharmaceutical tablet comprising a plurality of active pellets as defined in claim 11 and conventional tabletting excipients.
